(19) United States
(12) Patent Application Publication
(10) Pub. No.: US 2025/0000458 A1
(43) Pub. Date: Jan. 2, 2025

(54) STRETCH BAND WITH INDICATORS OR LIMITERS

(71) Applicant: MASIMO CORPORATION, Irvine, CA (US)

(72) Inventors: Yassir Kamel Abdul-Hafiz, Irvine, CA (US); Stephen Scruggs, Newport Beach, CA (US); Chad A. DeJong, Los Angeles, CA (US); Nicholas Evan Barker, Laguna Beach, CA (US); Kyla Yoland Scott, Huntington Beach, CA (US)

(21) Appl. No.: 18/767,277

(22) Filed: Jul. 9, 2024

Related U.S. Application Data
(63) Continuation of application No. 16/601,429, filed on Oct. 14, 2019, now abandoned.
(60) Provisional application No. 62/746,308, filed on Oct. 16, 2018.

(51) Int. Cl. A61B 5/00

(52) U.S. Cl. CPC A61B 5/6843; A61B 5/6803; A61B 5/6831; A61B 5/684

(57) ABSTRACT

A band configured to apply a force to a physiological sensor on a portion of a user's body can include an elastic segment, a first indicator, and a second indicator. The elastic segment can have a first end and a second end opposite the first end, and a first portion of the elastic segment can be secured to a second portion of the elastic segment to form a closed loop that can at least partially secure the physiological sensor to the portion of the user's body when in use. The first and second indicators can be spaced apart from one another and positioned along the elastic segment. The first indicator can be positioned closer to the first end than the second indicator. A relative distance between the first and second indicators can provide an indication of a desired stretch of the elastic segment.

### INCORPORATION BY REFERENCE TO ANY PRIORITY APPLICATIONS

This application is a continuation of U.S. patent application Ser. No. 16/601,429, entitled “STRETCH BAND WITH INDICATORS OR LIMITERS,” filed Oct. 14, 2019, which claims the benefit of priority under 35 U.S.C. § 119 (e) to U.S. Patent Application No. 62/746,308, entitled “HEADBAND WITH STRETCH INDICATORS OR LIMITERS,” filed Oct. 16, 2018, each of which is hereby incorporated by reference herein in its entirety.

### TECHNICAL FIELD

The present disclosure relates to bands that secure and/or wrap around a portion of a user's body to secure a physiological sensor.

### BACKGROUND

Various headband devices that can be used for athletic or medical-related purposes are known. Some headbands can be used to secure sensors or other devices to a user's head. Some headbands can be used to apply pressure to a region of a user's body proximate the headband and/or to secure a physiological sensor to a user's forehead. Applying an appropriate amount of force and/or pressure to a physiological sensor on a user's forehead can be important to increase the accuracy of measurement obtained by the sensor. At the same time, the applied force and/or pressure should be optimized so that the headband does not provide discomfort or injury to the wearer. There is a need for improved headband devices that provide better indications of a desired (for example, optimal) stretch of the headband when secured to a portion of a user's body (for example, forehead) and/or around a physiological sensor so that an appropriate amount of force and/or pressure is applied.

### SUMMARY

For purposes of summarizing the disclosure, certain aspects, advantages and novel features of several devices, systems, and methods have been described herein. It is to be understood that not necessarily all examples of the present disclosure are disclosed herein. Thus, the devices, systems, and methods disclosed herein can be embodied or carried out in a manner that achieves or optimizes one advantage or group of advantages as taught herein without necessarily achieving other advantages as can be taught or suggested herein. Disclosed herein is a band for securing a physiological sensor to a measurement site on a patient. The band provides a guide that allows a user to optimize an applied pressure by providing indications to guide the user to provide the correct amount of stretch on the band for a given patient.

Aspects of the present disclosure relate to a band configured to apply a force to a physiological sensor on a portion of a user's body. The band can comprise an elastic segment having a first end and a second end opposite the first end, a first indicator, and a second indicator. A first portion of the elastic segment can be configured to be secured to a second portion of the elastic segment to form a closed loop configured to at least partially secure the physiological sensor to the portion of the user's body when in use. The first and second indicators can be spaced apart from one another and positioned along the elastic segment. The first indicator can be positioned closer to the first end than the second indicator, and a relative distance between the first and second indicators can provide an indication of a desired stretch of the elastic segment. The first and second indicators can be positioned closer to the first end of the elastic segment than to the second end of the elastic segment. The band can further comprise a tab connected to the elastic segment proximate the first end. The tab can be configured to secure the first portion to the second portion. The elastic segment can comprise a first edge and a second edge opposite the first edge, and each of the first and second edges can extend along a length of the elastic segment between the first and second ends. Each of the first and second indicators can be positioned along the first edge. One or both of the first and second indicators can comprise a notch. The notch can comprise a half-circle shape. The elastic segment can further comprise a third indicator and a fourth indicator. The third and fourth indicators can be spaced apart from one another and positioned along the second edge of the elastic segment closer to the first end than to the second end. The third indicator can be positioned closer to the first end than the fourth indicator. The first, second, third, and fourth indicators can be configured to provide the indication of the optimal stretch of the elastic segment. The third indicator can be aligned with the first indicator and/or the fourth indicator can be aligned with the second indicator. The band can be a headband and the portion of the user's body can be a forehead of the user. The first and second indicators can be spaced apart from one another by a spacing. The spacing can be between 1% and 5% of a length of the clastic segment. The first and second indicators can be spaced apart from one another by a spacing such that extension of the elastic segment by an amount equal to the spacing results in a tensioning force between 0.10 lbf to 0.30 lbf. The first and second indicators can be spaced apart from one another by a spacing such that extension of the elastic segment by an amount equal to the spacing results in a pressure between 5 mmHg (0.0967 psi) and 15 mmHg (0.290 psi).

Aspects of the present disclosure relate to a method of securing a band around a physiological sensor on a portion of a user's body. The method can comprise wrapping the band around the portion of the user's body to at least partially cover the physiological sensor, wherein the band comprises: an elastic segment having a first end and a second end opposite the first end, the elastic segment having a first length when in an un-stretched position; and a first indicator and a second indicator, the first and second indicators spaced apart from one another and positioned along the elastic segment closer to the first end than to the second end, wherein the first indicator is positioned closer to the first end than the second indicator. The method can further comprise: at least partially aligning the first indicator with the second end of the elastic segment; stretching the band to a second length, the second length being greater than the first length; at least partially aligning the second indicator with the second end of the elastic segment; and securing the band to the portion of the user's body. The band can further comprise a tab connected to the elastic segment proximate the first end, and the step of securing the band to the portion of the user's body can comprise securing the tab to a portion of the elastic segment. The elastic segment can comprise a first edge and a second edge opposite the first edge, each of the first and second edges extending between the first and second ends of the elastic segment, and each of the first and second indicators can be positioned along the first edge. The first indicator can be a first notch and the second indicator can be a second notch, and the step of at least partially aligning the second end of the elastic segment with the first indicator can comprise positioning a portion of the second end within a space defined by the first notch. The step of at least partially aligning the second end of the elastic segment with the second indicator can comprise positioning the portion of the second end within a space defined by the second notch. The band can further comprise a third indicator and a fourth indicator. The third and fourth indicators can be spaced apart from one another and positioned along the second edge of the elastic segment closer to the first end than to the second end. The third indicator can be aligned with the first indicator and the fourth indicator can be aligned with the second indicator. The method can further comprise at least partially aligning the second end of the elastic segment with the third indicator simultaneously with the step of at least partially aligning the second end of the elastic segment with the first indicator. The method can further comprise at least partially aligning the second end of the elastic segment with the fourth indicator simultaneously with the step of at least partially aligning the second end of the elastic segment with the second indicator. The first and second indicators can be spaced apart from one another by a distance, and wherein the distance is between 1% and 5% of a length of the elastic segment. The first and second indicators can be spaced apart from one another by a spacing such that the step of stretching the band to the second length and at least partially aligning the second indicator with the second end of the elastic segment causes the elastic segment to exert a pressure on the physiological sensor between 5 mmHg (0.0967 psi) and 15 mmHg (0.290 psi).

Aspects of the present disclosure relate to a band configured to apply a force to a physiological sensor on a portion of a user's body. The band can comprise: an elastic segment having a first end and a second end opposite the first end; a tab connected to the elastic segment proximate the first end, the tab configured to secure to a portion of the elastic segment to form a closed loop around at least a portion of the physiological sensor and the portion of the user's body when in use; and a first plurality of notches positioned along a first edge of the clastic segment, each of the first plurality of notches spaced apart from one another by a pre-determined first spacing such that extension of the elastic segment by an amount equal to the first spacing results in a tensioning force between 0.10 lbf to 0.30 lbf. The first plurality of notches can be spaced apart from one another by the pre-determined first spacing such that extension of the elastic segment by the amount results in a tensioning force between 0.120 lbf to 0.240 lbf. The band can further comprise a second plurality of notches positioned along a second edge of the elastic segment and spaced apart from one another by the pre-determined first spacing, wherein the second edge is opposite the first edge. Each of the second plurality of notches can be aligned with a respective one of the first plurality of notches. Each of the first and second plurality of notches can comprise a half-circle shape.

Aspects of the present disclosure relate to a band configured to apply a force to a physiological sensor on a portion of a user's body, the band comprising: an elastic segment having a first end and a second end opposite the first end, wherein a first portion of the elastic segment is configured to be secured to a second portion of the elastic segment to form a closed loop around at least a portion of the physiological sensor and the portion of the user's body when in use; and a first indicator and a second indicator, the first and second indicators spaced apart from one another and positioned along the elastic segment, wherein the first indicator is positioned closer to the first end than the second indicator, and wherein a relative distance between the first and second indicators provides an indication of a desired stretch of the elastic segment.

Aspects of the present disclosure relate to a headband for applying a force to a physiological sensor on a forehead of a user can comprise and elastic segment and at least one indicator. The elastic segment can have a first end and a second end opposite the first end. In some embodiments, the headband also comprises a tab having a first end and a second end opposite the first end and the first end of the tab is connected to the elastic segment proximate the first end of the elastic segment. The tab can secure to a portion of the elastic segment and form a closed loop around the physiological sensor and the forehead of the user (for example, a patient). The at least one indicator can be positioned on a surface of the elastic segment between the first and second ends. The at least one indicator can be configured to indicate an extension of the elastic segment when stretched and/or a degree of stretch of the elastic segment. The at least one indicator can comprise a first shape when the elastic segment is in an un-stretched position and a second shape when the elastic segment is in an optimally-stretched position. The second shape can be different than the first shape. When the elastic segment is in the optimally-stretched position, the elastic segment can be configured to apply a predetermined pressure and/or force to the physiological sensor and a corresponding predetermined pressure and/or force can be applied to the forehead of the user via contact with the physiological sensor. The first shape can comprise a rectangle and the second shape can comprise a square. The rectangle can be oriented on the surface of the elastic segment such that long sides of the rectangle are perpendicular to an axis extending from the first end of the elastic segment to the second end of the elastic segment. The first shape can comprise an ellipse and the second shape can comprise a circle. Alternatively, the first shape can comprise a circle and the second shape can comprise an ellipse. The ellipse can be oriented on the surface of the clastic segment such that a major axis of the ellipse is perpendicular to an axis extending from the first end of the elastic segment to the second end of the elastic segment. The at least one indicator can comprise three indicators spaced equally (or un-equally) along a portion of the surface of the elastic segment. The at least one indicator can be laser drawn on the surface of the elastic segment. The at least one indicator can be positioned on the surface of the elastic segment using a colored ink. The at least one indicator can be positioned equidistant from opposite sides or edges of the elastic segment to increase accuracy of visual inspection of the indicator when the elastic segment is stretched. The first shape or the second shape of the at least one indicator can comprise an outer rectangle including a plurality of rectangles, and the plurality of rectangles can be positioned adjacent to one another and/or within the outer rectangle. The first shape or the second shape of the at least one indicator can comprise an outer square including a plurality of squares and the plurality of squares can be positioned adjacent to one another and/or within the outer square. The plurality of rectangles and/or plurality of squares can be hatched and/or shaded.

Aspects of the present disclosure relate to a method of securing a headband over a physiological sensor around a forehead of a user can comprise wrapping the headband around the forehead of the user and at least partially covering the physiological sensor. The headband can comprise an elastic segment having a first end and a second end opposite the first end. The headband can additionally comprise a tab having a first end and a second end opposite the first end. The first end of the tab can be connected to the elastic segment proximate the first end of the elastic segment and the at least one indicator can be positioned on a surface of the clastic segment between the first and second ends. The at least one indicator can be configured to indicate an extension of the elastic segment when stretched, wherein the at least one indicator comprises a first shape when the elastic segment is in an un-stretched position and a second shape when the elastic segment is stretched to an optimally-stretched position. The method can further comprise stretching the headband to the optimally-stretched position and attaching the tab (for example, a portion thereof) to a portion of the clastic segment to form a closed loop around the physiological sensor and the forehead of the user. The method can further comprise determining whether the at least one indicator has changed from the first shape to the second shape. The step of stretching the headband to the optimally-stretched position can occur simultaneously or non-simultaneously with the step of determining whether the at least one indicator has changed from the first shape to the second shape.

Aspects of the present disclosure relate to a headband for applying a force to a physiological sensor on a portion of a user's body can comprise an elastic segment having a first end and a second end opposite the first end. The headband can additionally comprise a tab having a first end and a second end opposite the first end. The first end of the tab can be connected to the elastic segment proximate to the first end of the elastic segment and the tab can be configured to secure to a portion of the elastic segment and form a closed loop around the physiological sensor and the portion of the user's body. The headband can additionally comprise at least one limiter positioned on a surface of the elastic segment between the first and second ends. The at least one limiter can be configured to limit an extension of the elastic segment. The at least one limiter can comprise one, two, three, four, five, six, seven, or eight or more limiters configured to limit an extension of the elastic segment. The at least one limiter can comprise a loose segment having a first end and a second end opposite the first end, both ends of the loose segment connected to portions of the surface of the elastic segment, and wherein the loose segment is slack when the elastic segment is in an un-stretched positioned and taut when the elastic segment is in an optimally-stretched position.

### BRIEF DESCRIPTION OF THE DRAWINGS

Various examples will be described hereinafter with reference to the accompanying drawings. These examples are illustrated and described by example only, and are not intended to limit the scope of the disclosure. In the drawings, similar elements have similar reference numerals.

FIG. 1A illustrates a physiological sensor attached or placed on a forehead of a user.

FIG. 1B illustrates a physiological sensor at least partially secured and/or covered with a band to a forehead of a user in accordance with aspects of this disclosure.

FIG. 2A illustrates a band having one or more indicators in accordance with aspects of this disclosure.

FIG. 2B illustrates the band of FIG. 2A in a stretched position in accordance with aspects of this disclosure.

FIG. 3A illustrates a band having one or more indicators in accordance with aspects of this disclosure.

FIG. 3B illustrates the band of FIG. 3A in a stretched position in accordance with aspects of this disclosure.

FIG. 4A illustrates a band having a stretch limiter in accordance with aspects of this disclosure.

FIG. 4B illustrates the band of FIG. 4A in a stretched position in accordance with aspects of this disclosure.

FIG. 5A illustrates a band having one or more indicators in accordance with aspects of this disclosure.

FIG. 5B illustrates the band of FIG. 5A in a stretched position in accordance with aspects of this disclosure.

FIG. 6A-6B illustrate various locations where a physiological sensor and a band can be secured to a user in accordance with aspects of this disclosure.

FIG. 7A illustrates a band having one or more indicators in accordance with aspects of this disclosure.

FIG. 7B illustrates a band having one or more indicators in accordance with aspects of this disclosure.

FIG. 7C illustrates a band having one or more indicators in accordance with aspects of this disclosure.

FIGS. 8A-8C illustrate a method of securing the band of FIG. 7A to a user's forehead in accordance with aspects of this disclosure.

FIGS. 9A-9C illustrate exemplary flow charts describing methods of securing a band to a portion of a user's body and applying a force and/or pressure to a sensor positioned on the portion of the user's body in accordance with aspects of this disclosure.

### DETAILED DESCRIPTION

The present disclosure will now be described with reference to the accompanying figures, wherein like numerals refer to like elements throughout. The following description is merely illustrative in nature and is in no way intended to limit the disclosure, its application, or uses. It should be understood that steps within a method may be executed in different order without altering the principles of the present disclosure. Furthermore, the devices, systems, and/or methods disclosed herein can include several novel features, no single one of which is solely responsible for its desirable attributes or which is essential to practicing the devices, systems, and/or methods disclosed herein.

Disclosed herein are embodiments of a band (also referred to herein as a “stretch band”) having one or more indicators and/or one or more limiters that can assist a wearer and/or a caregiver in appropriately extending (for example, “stretching”) the band so that a desired and/or optimal force and/or pressure is applied to a physiological sensor positioned and/or secured to a portion of the wearer's body, such as on the forehead of the wearer. As discussed elsewhere herein, in some cases the bands discussed herein can be used to secure the physiological sensor to the portion of the user's body (for example, where the sensor does not have an adhesive surface that can be attached to skin of the user). Advantageously, the application of such optimal force and/or pressure can prevent injury and/or discomfort to a portion of the user's body from the band when in use and can also allow a physiological sensor to more accurately determine physiological parameters from the portion of the user's body. While the present disclosure often uses the term “headband,” the use of such term is not intended to mean that the disclosed devices, systems, and/or methods can only be used in relation to a user's head or portion thereof. Rather, as discussed below, the devices, systems, and/or methods disclosed herein can be used to apply a desired and/or optimal force and/or pressure to a physiological sensor that can be placed or secured to any portion of a user's body, including but not limited to a user's head.

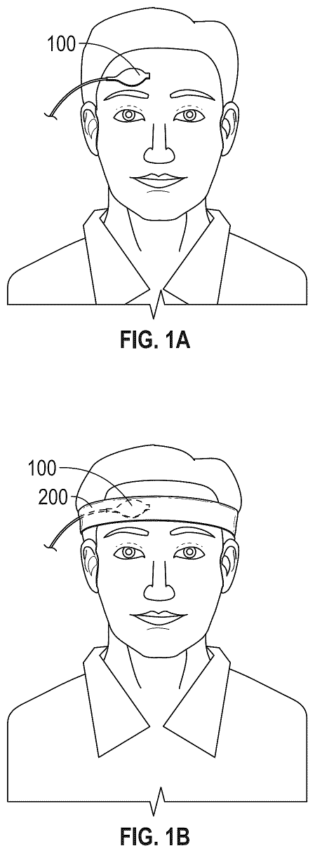

FIG. 1A illustrates a physiological sensor 100 attached and/or placed on a forehead of a user. The physiological sensor 100 can be an oximetry sensor including one or more emitters and/or one or more detectors configured to emit light into tissue and detect light attenuated through the tissue. For example, the physiological sensor 100 can be a regional oximetry sensor that operates in a reflectance mode whereby the emitter(s) and detector(s) are located on the same side of a user's body. Measurements from the physiological sensor 100 can be used to detect oxygen saturation arterial blood hemoglobin (SpO2) and/or pulse rate, for example. The physiological sensor 100 can include a cable and/or connector which can connect to a cable, either of which may be used to couple the physiological sensor 100 to a physiological monitoring system or patient monitoring device that can process and/or display information related to the measurements obtained from the physiological sensor 100, among other things. One example of a physiological sensor 100 is a regional oximetry sensor described in U.S. Patent Publication No. 2015/0099950 assigned to Masimo Corporation of Irvine, CA, the entirety of which is hereby incorporated by reference. However, physiological sensor 100 can be any sensor configured to obtain physiological measurements from a user, such as an electrocardiogram (ECG) sensor, electroencephalography (EEG) sensor, a pulse rate sensor, an acoustic sensor, an electromechanical sensor, among others.

FIG. 1B illustrates a headband 200 secured to a portion of a user's forehead around the physiological sensor 100. The headband 200 can be placed around, can at least partially cover, and/or can secure a portion of the physiological sensor 100 to the user's forehead or can secure the entirety of the physiological sensor 100 to the user's forehead. When the headband 200 is secured to the forehead of the user (for example, by attachment of one end of the headband 200 to an opposite end of the headband 200), the headband 200 can cover a portion of the physiological sensor 100 or can cover the entirety of the physiological sensor 100 (see FIG. 1B). For example, in some cases the headband 200 can have a width that is greater than a width of the physiological sensor 100 so that the headband 200 covers the entirety of the physiological sensor 100 when placed overtop or alongside the physiological sensor 100 (see FIG. 1B). The physiological sensor 100 can be secured to the forehead of the user by an adhesive on a surface of the sensor 100 and be additionally secured to the user's forehead by the headband 200. In some cases, the physiological sensor 100 does not have an adhesive and is secured to the user's forehead only via the headband 200. The headband 200 can secure the physiological sensor 100 to the forehead of the user (or another portion of the user's body) by application of a force and/or pressure to the physiological sensor 100 when secured to and/or around the user's forehead (or other portion of the user's body). The force and/or pressure applied to the physiological sensor 100 by the headband disclosed herein can advantageously provide for more accurate measurements taken by the physiological sensor 100, as discussed further below. While FIGS. 1A and 1B are shown and discussed primarily with reference to headband 200, it is understood that headband 300 (discussed further below) can be used in the same or a similar way to secure to a forehead of a user and/or to apply a force and/or pressure to sensor 100.

The force and/or pressure that is applied to the physiological sensor 100 by the bands disclosed herein (for example, headband 200, 300) can be an important aspect in the proper functioning and/or measurement capabilities of the physiological sensor 100. For example, in some situations, measurements of the physiological sensor 100 may be subject to errors and/or inaccuracies depending on conditions and/or characteristics of a user. Such situations can include, without limitation: when the user is lying down with his/her head near or below his/her chest; when the user is experiencing elevated venous pressure; when the user is diaphoretic; when the user is at risk or predisposed to experiencing involuntary movements, such as seizures, or when the user is moving excessively, such as during exercise activities; and/or other situations when venous pulsations can result in errors in oximetry measurements. In these situations, such venous pulsations may reduce the accuracy of physiological measurements (for example, oxygen saturation and/or pulse rate measurements). The bands disclosed herein (such as headband 200, 300) can apply force and/or pressure to the physiological sensor 100 and therefore reduce such effects. Advantageously, the pressure applied by the bands disclosed herein (such as headband 200, 300) can be greater than venous pressure so as to reduce the effects discussed above, but less than some predetermined value so as to not cause injury and/or so as to not prevent meaningful measurements (for example, so as not to cut off blood flow in and/or around region proximate the sensor 100). As an example, the pressure applied by the bands disclosed herein to the physiological sensor 100 and/or the region of a user's skin adjacent to the sensor 100 can be less than arteriole pressure. The application of force and/or pressure over a physiological sensor 100 can improve arterial hemoglobin oxygen saturation reading accuracy in the presence of venous pooling and/or pulsations are the site of the user's body where the sensor 100 is obtaining measurements.

In some cases, the bands discussed herein (such as headband 200, 300) can be configured to apply a predetermined desired and/or optimal pressure that is greater than a first threshold (for example, representing venous pressure) and at the same time less than a second threshold (for example, representing a maximum comfort force and/or pressure). As discussed further below, the headband 200, 300 can include various indicators and/or limiters that help a wearer and/or caregiver determine how much to extend (for example, stretch) the headband 200, 300 so that the headband 200, 300 applies a given force onto physiological sensor 100 that in turn results in a pressure on a portion of the wearers body (for example, underneath the surface of the sensor 100). Such resulting force can be, for example, 0.060 lbf, 0.120 lbf, 0.180 lbf, 0.240 lbf, 0.300 lbf, 0.360 lbf, 0.420 lbf, or 0.480 lbf, or any value therebetween, or any range bounded by any combination of these values, although values outside these values or ranges can be used in some cases. As another example, the headband 200, 300 can include various indicators and/or limiters that help a wearer and/or caregiver determine how much to extend (for example, stretch) the headband 200, 300 so that the headband 200, 300 applies a given pressure to and/or around the sensor 100 and/or to a portion of the user's body. For example, the pressure can be 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi), or any value there between, or any range bounded by any combination of these values, although values outside these values or ranges can be used in some cases. Utilizing the one or more indicators 202, 204, 220, 304a, 304b, 314a, 314b, 324 and/or the one or more stretch limiters 206, a wearer and/or caregiver can extend (for example, stretch) the headband 200, 300 to a desired and/or optimal stretched length to selectively apply a force and/or pressure to the sensor 100 and/or the portion of the user's body such as those mentioned above.

The bands discussed herein (such as bands 200, 300) can be adjusted for use with any size wearer by using an adjustable closure mechanism, such as a hook and loop closure mechanism or an adhesive securement mechanism. Such adjustable closure mechanisms can be located along one or more portions of the interior and/or exterior the bands 200, 300, such as at one or both ends of the bands 200, 300. For example, a portion of the bands 200, 300 can secure to another portion of the headband 200, 300 via a hook and loop closure mechanism and/or an adhesive. As another example, a portion of an inner surface of the headband 200, 300 proximate a first end of the headband 200 can secure to a portion of an outer surface of the bands 200, 200 via a hook and loop closure mechanism or an adhesive, and such securement can result in the bands 200, 300 forming a closed loop around a physiological sensor 100 (or a portion thereof) and/or a portion of a user's body (for example, forehead). The bands discussed herein (such as bands 200, 300) can have varying dimensions and/or sizes based on the shapes and sized of the wearer. For example, where the bands discussed herein are used to wrap around and/or secure to a user's forehead, the bands can have varying dimensions and/or sizes based on the size of a wearer's forehead. For example, the bands 200, 300 can have a generally smaller size where used for neonatal patients and can have a generally larger size when used for an adult. Wearers of the bands 200, 300 can apply a wide range of force and/or pressures to the physiological sensor 100 depending on the amount of stretch and/or tension that is applied to the bands 200, 300 before, during, or after placement around the portion of the wearer's body to which the bands 200, 300 are secured. The bands discussed herein (such as headbands 200, 300) can eliminate or significantly reduce the need for “estimation” by caregivers who may be applying the bands 200, 300 to a user's forehead (or other portion of the user's body) by providing them with an indication of the appropriate amount of extension (for example, “stretch”) necessary in the bands 200, 300 in order to apply a desired and/or optimal amount of force and/or pressure to the sensor 100 and/or the tissue underneath, adjacent, and/or proximate to the sensor 100.

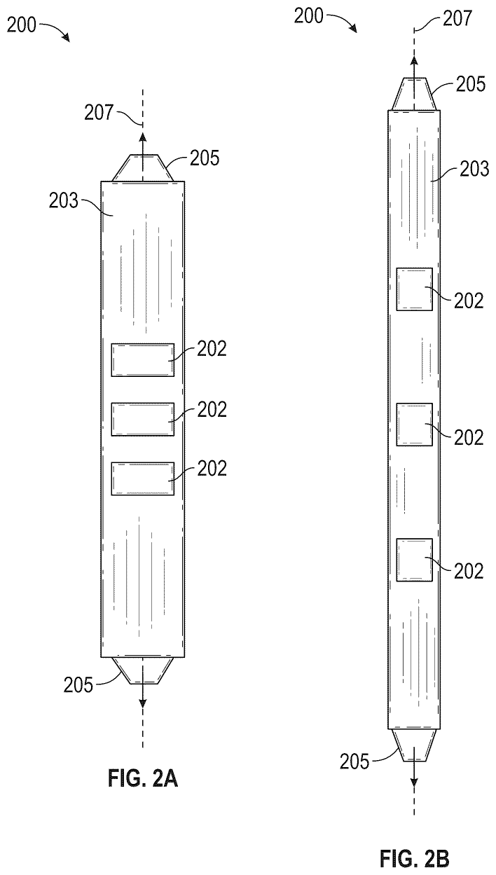

With reference to FIGS. 2A-5B, the headband 200 can have one or more indicators and/or limiters that can assist a wearer and/or caregiver in applying the headband 200 to a portion of the wearer's body (for example, the forehead). More specifically, the one or more indicators and/or limiters can help the wearer and/or caregiver stretch the headband 200 an appropriate amount to achieve a desired and/or optimal pressure and/or force to the sensor 100 and/or portion of the wearer's body underneath, adjacent, and/or proximate the sensor 100. The headband 200 can include an elastic segment 203 that extends from one end of the headband 200 to an opposite end of the headband 200. The headband 200 can have a tab 205 positioned and/or attached to the elastic segment 203 at or proximate to one or both ends of the headband 200. The tab 205 can have an adjustable closure mechanism, such as a hook and loop closure mechanism or adhesive securement mechanism which can cooperate and/or engage with adjustable closure mechanism(s) on another portion of the headband 200 (for example, the elastic segment 203). Alternatively, the headband 200 can have no tab 205, but rather, can include the adjustable closure mechanism on and/or proximate to one or both ends of the elastic segment 203 for securing each end to one another, for example. The tab 205 can be made of a different material than the elastic segment 203 of the headband 200. For example, tab 205 can be made of a material that is not clastic, so that the tab 205 can be used to pull and/or stretch the elastic segment 203 without itself stretching. In some cases, the tab 205 can be welded or otherwise permanently attached to a portion of the elastic segment 203.

FIGS. 2A-2B show a side view of a face or surface of a headband 200 having one or more indicators 202 (also referred to as “stretch indicators 202”). As discussed in more detail below, the one or more indicators 202 can provide an indication of an amount of stretch of the headband 200 which corresponds and/or achieves a desired force and/or pressure, for example, on a physiological sensor 100 and/or a portion of a user's body adjacent the sensor 100 when the headband 200 is secured thereto. Headband 200 can include one or more indicators 202, such as one, two, three, four, five, six, seven, or eight or more indicators 202. The indicators 202 can be placed on a surface of the elastic segment 203 at various locations along a length of the elastic segment 203 of the headband 200. The one or more indicators 202 can be spaced along the length of the elastic segment 203 of the headband 200 at various distances and/or spacings. For example, the indicators 202 can be spaced equally from each other. Including a plurality of indicators 202 spaced from each other along the length of elastic segment 203 can allow for visual inspection of the indicators 202 from varying viewpoints with respect to the portion of the wearer's body where the headband 200 is secured. For example, positioning the indicators 202 in a middle portion of the headband 200 meant to be at or near a forehead region of the wearer. Positioning additional indicators 202 along the clastic segment 203 between ends of the headband 200 meant to be at or near sides of the head of the wearer allows the wearer and/or caregiver to inspect the indicators 202 from different positions.

The one or more indicators 202 can comprise various shapes or objects that can change when the headband 200 is stretched. The indicators 202 comprising shapes or objects can be oriented so that their outline or shape can vary according to a predetermined scheme or method when the headband 200 is stretched. As shown by FIG. 2A, indicator 202 can comprise a rectangle shape when the headband 200 is in an un-stretched position. The indicators 202 can be oriented so that, when the headband 200 is stretched, the shape of the indicator 202 changes from a rectangle shape (see FIG. 2A) to a square shape (see FIG. 2B). The headband 200 can be extended and/or stretched in a direction parallel to an axis 207 extending through a length of the headband 200 (see arrows in FIGS. 2A-2B). Such stretching or extension in this direction can cause the headband 200 to become narrower and longer via clastic behavior of the headband 200. FIG. 2A shows the headband 200 in an un-stretched position with a smaller length, but greater width, than the headband 200 in the stretched position in FIG. 2B. Such stretching of the headband 200 can cause the cross-section of the headband 200 to decrease, which in turn can cause the indicators 202 to change in shape from a rectangle shape to a square shape. The rectangle indicator(s) 202 can be oriented such that, when headband 200 is in an un-stretched position, longer sides of the rectangles are perpendicular to the axis 207 extending through the length of the headband 200.

The one or more indicator 202 can be used to aid a wearer or a caregiver to appropriately stretch the headband 200 so that a desired or optimal pressure is applied to a physiological sensor 100 and/or a region of the wearer's body underneath, adjacent, and/or proximate the sensor 100. For example, when applying the headband 200 to the wearer, the wearer and/or caregiver can stretch the headband 200 a given amount until the indicator(s) 202 transform from one shape to another shape. Once the wearer and/or caregiver notices such transformation, the wearer and/or caregiver can secure the headband 200 to the wearer's body (for example, by attaching an end or tab of the headband 200 to another portion of the headband 200). Such technique can advantageously provide a quick and simple method by which the wearer and/or caregiver can ensure that the headband 200 is secured to the wearer and/or the physiological sensor 100 with an optimal applied pressure. The one or more indicators 202 can be designed, shaped, and/or otherwise configured so that the headband 200 exerts a desired or optimal force and/or pressure when stretched an amount which causes a transformation of the one or more indicators 202 from a first shape (for example, rectangle) to a second shape (for example, a square). For example, when headband 200 is stretched an amount that causes a transformation of an indicator 202 from a rectangular shape to a square shape, the headband 200 can apply a force of 0.060 lbf, 0.120 lbf, 0.180 lbf, 0.240 lbf, 0.300 lbf, 0.360 lbf, 0.420 lbf, or 0.480 lbf on a sensor 100 and/or a forehead of a user, or any value therebetween, or any range bounded by any combination of these values, although values outside these values or ranges can be used in some cases. As another example, when headband 200 is stretched an amount that causes a transformation of an indicator 202 from a rectangular shape to a square shape, the headband 200 can apply a pressure of 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi) on a sensor 100 and/or a forehead of a user, or any value therebetween, or any range bounded by any combination of these values, although values outside these values or ranges can be used in some cases.

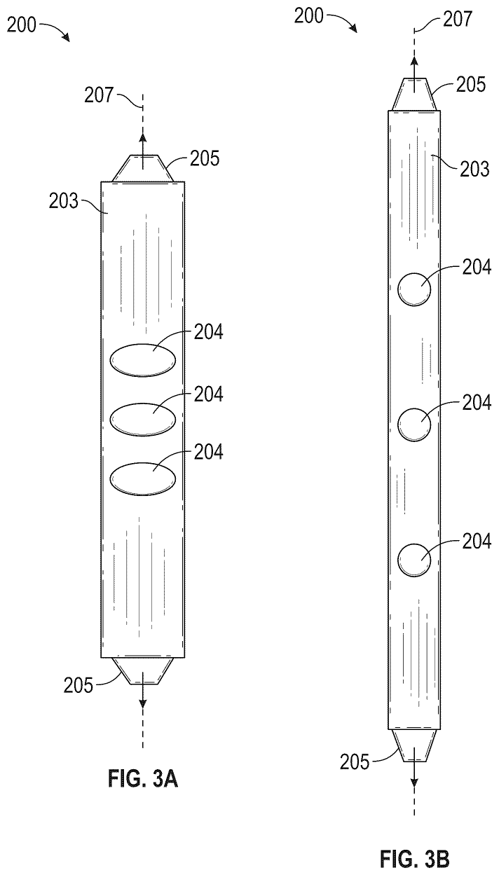

FIGS. 3A-3B show additional indicators 204 that can be used in similar manner as indicators 202. FIGS. 3A-3B show a side view of a face or surface of headband 200 having indicators 204 (also referred to as “stretch indicators 204”). As discussed in more detail below, the one or more indicators 204 can provide an indication of an amount of stretch of the headband 200 which corresponds and/or achieves a desired force and/or pressure, for example, on a physiological sensor 100 and/or a portion of a user's body adjacent the sensor 100 when the headband 200 is secured thereto. Headband 200 can include one or more indicators 204, such as one, two, three, four, five, six, seven, or eight or more indicators 204. The one or more indicators 204 can be placed on a surface of the elastic segment 203 at various locations along the length of the elastic segment 203 of the headband 200. The one or more indicators 204 can be spaced along the length of the elastic segment 203 of the headband 200 at various distances and/or spacings. For example, the indicators 204 can be spaced equally from each other. Including a plurality of indicators 204 spaced from each other along the length of elastic segment 203 can allow for visual inspection of the indicators 204 from varying viewpoints with respect to the portion of the wearer's body where the headband 200 is secured. For example, positioning the indicators 204 in a middle portion of the headband 200 meant to be at or near a forehead region of the wearer. Positioning additional indicators 204 along the elastic segment 203 between ends of the headband 200 meant to be at or near sides of the head of the wearer allows the wearer and/or caregiver to inspect the indicators 204 from different positions.

As shown by FIG. 3A, the one or more indicators 204 can comprise an elliptical shape when the headband 200 is in an un-stretched position. The indicators 204 can be oriented so that, when the headband 200 is stretched, the shape of the indicators 204 changes from an elliptical shape (see FIG. 3A) to a circle shape (see FIG. 3B). The headband 200 can be extended and/or stretched in a direction parallel to an axis 207 extending through a length of the headband 200 (see arrows in FIGS. 3A-3B). Such stretching or extension in this direction can cause the headband 200 to become narrower and longer via elastic behavior of the headband 200. FIG. 3A shows the headband 200 in an un-stretched position with a smaller length, but greater width, than the headband 200 in the stretched position. Such stretching of the headband 200 can cause the cross-section of the headband 200 to decrease, which in turn can cause the indicators 204 to change in shape from an elliptical shape to a circle shape. The indicators 204 can be oriented such that, when the headband 200 is in an un-stretched position (see FIG. 3A) major axes of the elliptical stretch indicators 204 are perpendicular to the axis 207 extending through the length of the headband 200.

Similar to as discussed with respect to indicators 202, indicators 204 can be used to aid a wearer or a caregiver to appropriately stretch the headband 200 so that a desired or optimal pressure is applied to a physiological sensor 100 and/or a region of the wearer's body underneath, adjacent, and/or proximate the sensor 100. For example, when applying the headband 200 to the wearer, the wearer and/or caregiver can stretch the headband 200 a given amount until the indicator(s) 204 transform from one shape to another shape. Once the wearer and/or caregiver notices such transformation, the wearer and/or caregiver can secure the headband 200 to the wearer's body (for example, by attaching an end or tab of the headband 200 to another portion of the headband 200). Such technique can advantageously provide a quick and simple method by which the wearer and/or caregiver can ensure that the headband 200 is secured to the wearer and/or the physiological sensor 100 with an optimal applied pressure. The one or more indicators 204 can be designed, shaped, and/or otherwise configured so that the headband 200 exerts a desired or optimal force and/or pressure when stretched an amount which causes a transformation of the one or more indicators 204 from a first shape (for example, ellipse) to a second shape (for example, a circle). For example, when headband 200 is stretched an amount that causes a transformation of an indicator 204 from an elliptical shape to a circular shape, the headband 200 can apply a force of 0.060 lbf, 0.120 lbf, 0.180 lbf, 0.240 lbf, 0.300 lbf, 0.360 lbf, 0.420 lbf, or 0.480 lbf on a sensor 100 and/or a forehead of a user, or any value therebetween, or any range bounded by any combination of these values, although values outside these values or ranges can be used in some cases. As another example, when headband 200 is stretched an amount that causes a transformation of an indicator 204 from an elliptical shape to a circular shape, the headband 200 can apply a pressure of 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi) on a sensor 100 and/or a forehead of a user, or any value therebetween, or any range bounded by any combination of these values, although values outside these values or ranges can be used in some cases.

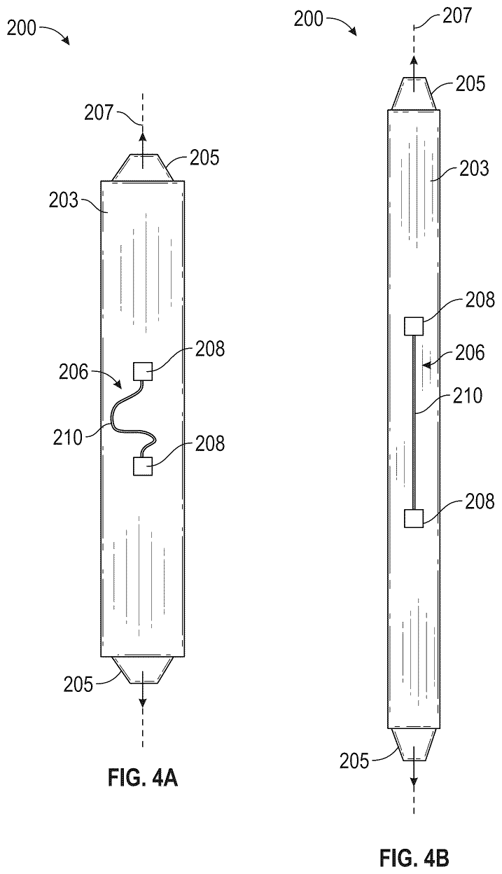

FIGS. 4A-4B show a side view of a face or surface of a headband 200 with a limiter 206 (also referred to as “stretch limiter 206”). As discussed in more detail below, the one or more limiters 206 can provide an indication of an amount of stretch of the headband 200 which corresponds and/or achieves a desired force and/or pressure, for example, on a physiological sensor 100 and/or a portion of a user's body adjacent the sensor 100 when the headband 200 is secured thereto. Limiter 206 can have a loose segment 210 and anchors 208 that secure opposite ends of the loose segment 210 to portions of the headband 200. In some cases, the limiter 206 includes a loose segment 210 without anchors 208, and ends of loose segment 210 can be secured to portions of the elastic segment 203. The loose segment 210 can be any suitable string or piece of fabric. While FIGS. 4A-4B illustrate a headband 200 having one limiter 206, the headband 200 can include more than one stretch limiter 206, such as two, three, or four or more limiters 206. The headband 200 can have a plurality of limiters 206 spaced vertically or horizontally with respect to one another along a length and/or width of the headband 200 (for example, the elastic segment 203). Such limiters 206 can be spaced along the length of the headband 200, similar to the spacing of the indicators 202, 204. Limiter 206 can be placed on a surface of the elastic segment 203 at various locations along the length and/or width of the elastic segment 203 of the headband 200. Anchors 208 can be a string or piece of fabric that secures the end of the loose segment 210 to portions of the elastic segment 203 of the headband 200. Alternatively, anchors 28 can be patches of fabric that secure the end of the loose segment 210 to portions of the elastic segment 203 of the headband 200 as illustrated in FIGS. 4A-4B. In some cases, the anchors 208 comprise a material that is configured not to stretch and/or change shape and/or size when the headband 200 is stretched. For example, the anchors 208 can comprise a different material and/or different material composition than the elastic segment 203 so that the anchors 208 are not elastic and/or are less elastic than the elastics segment 203. Alternatively, the anchors 208 can comprise a material that is configured to stretch and/or change shape and/or size when the headband 200 is stretched.

Limiter(s) 206 can be oriented so that, when the headband 200 is stretched, the slack of the loose segment 210 is reduced. For example, the headband 200 can be extended and/or stretched in a direction parallel to an axis 207 extending through the headband 200 (see arrows in FIGS. 4A-4B). Such stretching or extension in this direction can cause the headband 200 to become narrower and longer, via elastic behavior of the headband 200. FIG. 4A shows the headband 200 in an un-stretched position with a smaller length, but greater width, than the headband 200 in the stretched position as shown in FIG. 4B. Such stretching of the headband 200 can cause the cross-section of the headband 200 to decrease and portions of the headband 200 to translate outward toward the direction shown in the arrows in FIGS. 4A-4B. Further, because the loose segment 210 can be secured to portions of the headband via anchors 208, when the headband 200 stretches, the anchors 208 can move along with portions of the headband 200. As a result, slack in the loose segment 210 can be reduced until the loose segment 210 is straightened to its maximum length and becomes taut. Where the headband 200 does not include anchors 208, ends of loose segment 210 can be secured to portions of the headband 200 such that, when headband 200 stretches, the slack in the loose segment 210 can be reduced until the loose segment 210 is straightened to its maximum length and becomes taut.

Similar to as that discussed with reference to indicators 202, 204, limiter 206 can be used to aid a wearer or a caregiver to appropriately stretch the headband 200 so that a desired or optimal pressure is applied to a physiological sensor 100 and/or a region of the wearer's body underneath, adjacent, and/or proximate the sensor 100. For example, when applying the headband 200 to the wearer, the wearer and/or caregiver can stretch the headband 200 a given amount until the slack in the loose segment 210 of limiter 206 is reduced so that the loose segment 210 is taut. Once the wearer and/or caregiver notices the loose segment 210 reaches a maximum tightness and/or straightening, the wearer and/or caregiver can secure the headband 200 to the wearer's body (for example, by attaching an end or tab of the headband 200 to another portion of the headband 200). Such technique can advantageously provide a quick and simple method by which the wearer and/or caregiver can ensure that the headband 200 is secured to the wearer and/or the physiological sensor 100 with an optimal applied pressure. The one or more limiters 206 can be designed, shaped, and/or otherwise configured so that the headband 200 exerts a desired or optimal force and/or pressure when stretched an amount which causes the limiter 206 (and/or loose segment 210) to reach a maximum tightness and/or straightening. For example, when headband 200 is stretched an amount that causes the limiter 206 (and/or loose segment 210) to reach a maximum tightness and/or straightening, the headband 200 can apply a force of 0.060 lbf, 0.120 lbf, 0.180 lbf, 0.240 lbf, 0.300 lbf, 0.360 lbf, 0.420 lbf, or 0.480 lbf on a sensor 100 and/or a forehead of a user, or any value therebetween, or any range bounded by any combination of these values, although values outside these values or ranges can be used in some cases. As another example, when headband 200 is stretched an amount that causes the limiter 206 (and/or loose segment 210) to reach a maximum tightness and/or straightening, the headband 200 can apply a pressure of 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi) on a sensor 100 and/or a forehead of a user, or any value therebetween, or any range bounded by any combination of these values, although values outside these values or ranges can be used in some cases.

The loose segment 210 of the stretch limiter 206 can comprise a material that has little or no ability to elastically stretch. For example, unlike the elastic segment 203 which can comprise an elastic material which allows it to stretch elastically, the loose segment 210 can comprise a material that does not allow it to extend or stretch beyond its length. This can advantageously ensure that the stretch limiter 206 provides a proper gauge of the appropriate stretch of the headband 200 that corresponds with the optimal applied pressure. For example, the headband 200 can be stretched a maximum length so that the loose segment 210 is pulled taut and/or straightened fully, thus signaling to the wearer and/or the caregiver that the optimal stretch has been reached.

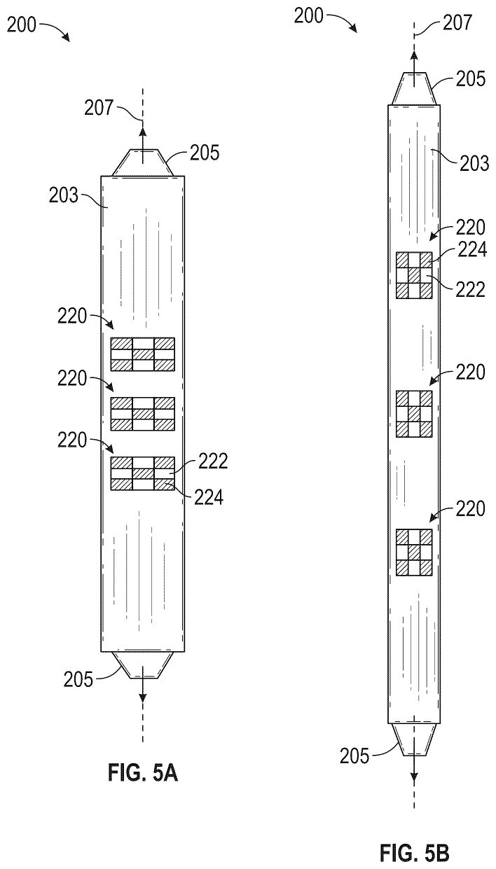

FIGS. 5A-5B illustrate one or more indicators 220 that can be used in similar manner as indicators 202, 204. FIGS. 5A-5B show a side view of a face or surface of headband 200 having indicators 220 (also referred to as “stretch indicators 220”). As discussed in more detail below, the one or more indicators 220 can provide an indication of an amount of stretch of the headband 200 which corresponds and/or achieves a desired force and/or pressure, for example, on a physiological sensor 100 and/or a portion of a user's body adjacent the sensor 100 when the headband 200 is secured thereto. Headband 200 can include one or more indicators 220, such as one, two, three, four, five, six, seven, or eight or more indicators 220. The one or more indicators 220 can be placed on a surface of the elastic segment 203 at various locations along the length of the elastic segment 203 of the headband 200. The one or more indicators 220 can be spaced along the length of the elastic segment 203 of the headband 200 at various distances and/or spacings. For example, the indicators 220 can be spaced equally from each other. Including a plurality of indicators 220 spaced from each other along the length of clastic segment 203 can allow for visual inspection of the indicators 220 from varying viewpoints with respect to the portion of the wearer's body where the headband 200 is secured. For example, positioning the indicators 220 in a middle portion of the headband 200 meant to be at or near a forehead region of the wearer. Positioning additional indicators 220 along the elastic segment 203 between ends of the headband 200 meant to be at or near sides of the head of the wearer allows the wearer and/or caregiver to inspect the indicators 220 from different positions.

As shown by FIG. 5A, indicators 220 can comprise a pattern, which itself can include one or more shapes. For example, indicators 220 can include a pattern of rectangles positioned adjacent to one another. The pattern of rectangles can include a plurality of rectangles adjacent to one another to form a larger rectangle. Some of the plurality of rectangles within the larger rectangle can be shaded, hatched, or otherwise differentiated from other rectangles within the larger rectangle. For example, as shown in FIG. 5A, the indicators 220 can comprise a pattern of nine rectangles, five of which are hatched (labeled as rectangles 224) and four of which are not hatched (labeled as rectangles 222). As shown, indicators 220 can comprise a pattern of rectangles when the headband 200 is in an un-stretched position. The pattern of rectangles of indicators 220 can be oriented so that, when the headband 200 is stretched, the rectangles change to squares (see FIGS. 5A-5B). The headband 200 can be extended and/or stretched in a direction parallel to an axis 207 extending through the headband 200 (see arrows in FIGS. 5A-5B). Such stretching or extension in this direction can cause the headband 200 to become narrower and longer, via elastic behavior of the headband 200. FIG. 5A shows the headband 200 in an un-stretched position with a smaller length, but greater width, than the headband 200 in the stretched position as shown in FIG. 5B. Such stretching of the headband 200 causes the cross-section of the headband 200 to decrease, which in turn can cause the indicators 220 to change in shape, from a rectangular pattern to a square pattern. The rectangular pattern of indicators 220 can be oriented such that, when headband 200 is in an un-stretched position, long sides of the rectangles are perpendicular to the axis 207 extending through the headband 200 (see FIG. 5A).

Similar to as discussed with respect to indicators 202, 204, indicator 220 can be used to aid a wearer or a caregiver to appropriate stretch the headband 200 so that a desired or optimal pressure is applied to a physiological sensor 100 and/or a region of the wearer's body underneath, adjacent, and/or proximate the sensor 100. For example, when applying the headband 200 to the wearer, the wearer and/or caregiver can stretch the headband 200 a given amount until the indicators 220 transform from one pattern of shapes to another pattern of shapes. Once the wearer and/or caregiver notices such transformation, the wearer and/or caregiver can secure the headband 200 to the wearer's body (for example, by attaching an end or tab of the headband 200 to another portion of the headband 200). Such technique can advantageously provide a quick and simple method by which the wearer and/or caregiver can ensure that the headband 200 is secured to the wearer and/or the physiological sensor 100 with an optimal applied pressure. The one or more indicators 220 can be designed, shaped, and/or otherwise configured so that the headband 200 exerts a desired or optimal force and/or pressure when stretched an amount which causes a transformation of the one or more indicators 220 from a first shape (for example, pattern of rectangles) to a second shape (for example, a pattern of squares). For example, when headband 200 is stretched an amount that causes a transformation of an indicator 220 from a partially-hatched pattern of rectangles to a partially-hatched pattern of squares, the headband 200 can apply a force of 0.060 lbf, 0.120 lbf, 0.180 lbf, 0.240 lbf, 0.300 lbf, 0.360 lbf, 0.420 lbf, or 0.480 lbf on a sensor 100 and/or a forehead of a user, or any value therebetween, or any range bounded by any combination of these values, although values outside these values or ranges can be used in some cases. As another example, when headband 200 is stretched an amount that causes a transformation of an indicator 220 from a partially-hatched pattern of rectangles to a partially-hatched pattern of squares, the headband 200 can apply a pressure of 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi) on a sensor 100 and/or a forehead of a user, or any value therebetween, or any range bounded by any combination of these values, although values outside these values or ranges can be used in some cases.

Advantageously, incorporating a pattern of shapes into indicators 220 can provide for more precise visual inspection of the degree and/or amount of stretch of the headband 200. For example, incorporating multiple rectangles within indicator 220 allows a wearer and/or caregiver to inspect each of the individual rectangles within the indicator 220 along a direction parallel and/or perpendicular to the axis 207 to determine whether the transformation from rectangle to square has in fact occurred (and thus optimal stretch amount has occurred). This precise visual inspection is aided where some of the rectangles are differentiated with hatching and/or shading, since such differentiation can allow users to more easily detect small changes between individual adjacent rectangles within the pattern of indicator 220 (for example, compare hatched rectangles 224 with non-hatched rectangles 222).

Further, incorporating a pattern of shapes within and/or as part of indicator 220 can allow wearers and/or caregivers to more precisely determine whether the headband 200 is stretched uniformly along axis 207. For example, if a wearer and/or caregiver pulls tabs 205 at sides of the tabs 205 (as opposed to a center portion of the tabs 205) some portions of the elastic segment 203 (for example, portions of elastic segment 203 to the right of the dotted axis 207) may be stretched more than other portions (for example, portions of elastic segment 203 to the left of the dotted axis 207). In such situations, some of the rectangles in the pattern of indicators 220 may appear different than other rectangles. This can indicate to a wearer and/or caregiver that a more uniform and/or centered tension needs to be applied along axis 207 so that the headband 200 can be properly secured and can apply uniform pressure to the sensor 100 when secured to a patient.

The indicators discussed herein (for example, indicators 202, 204, 220, and/or 324) can be positioned and/or placed on the headband 200 using various techniques. For example, the indicators 202, 204, 220, and/or 324 can be positioned on the headband 200 by being laser drawn. Alternatively, the indicators can be drawn on the headband 200 by inking, which can be advantageous in some situations where there is concern about damaging and/or reducing the structural integrity of the headband 200 materials.

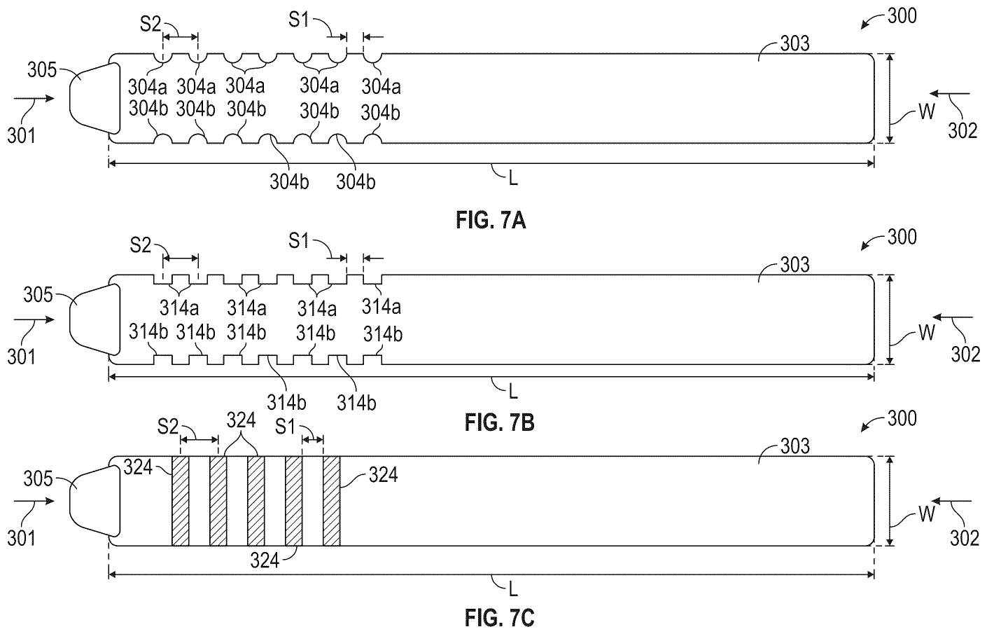

FIGS. 7A-7C illustrate additional embodiments of a headband 300. Headband 300 can include a length L, width W, first end 301, and a second end 302 opposite the first end 301. Headband 300 can include an elastic segment 303 and one or more tabs 305. Headband 300 can include a tab 305 secured to a portion of the elastic segment 303, for example, at or proximate to first end 301. Headband 300 can have one or more indicators configured to provide an indication of a stretch or extension of the headband 300. For example, as discussed in more detail below, the one or more indicators 304a, 304b, 314a, 314b, and/or 324 can provide an indication of an amount of stretch of the headband 300 which corresponds and/or achieves a desired force and/or pressure, for example, on a physiological sensor 100 and/or a portion of a user's body adjacent the sensor 100 when the headband 300 is secured thereto. As an example, when the headband 300 is wrapped around a portion of a user's body (for example, a forehead of the user) the location of the second end 302 with reference to one or more of the indicators discussed below (for example, indicators 304a, 304b, 314a, 314b, 324) can inform a user or a caregiver how much to stretch the headband 300 prior to securing the headband 300 to the portion of the user's body in order to achieve a desired or optimal force and/or pressure. Such indicators can be integral with the elastic segment 303 of headband 300, or alternatively, can be non-integral. For example, while indicators 304a, 304b, 314a, 314b, and 324 are illustrated in FIGS. 7A-7C as being integral with (for example, a part of) elastic segment 303, headband 300 can have indicators that are attached to elastic segment 303 after the elastic segment 303 is manufactured but which still provide an indication of a stretch or extension of the headband 300. For example, headband 300 can include indicators that are positioned and/or aligned as shown in FIGS. 7A-7C by indicators 304a, 304b, 314a, 314b, 324, but which are separate pieces or objects. For example, such indicators can comprise a separate material than the elastic segment 303 (for example, plastic) and can be secured to clastic segment 300.

As discussed above, headband 300 can include a tab 305 on one or both ends 301, 302 of headband 300. Tab(s) 305 can be permanently secured to a portion of headband 300 (for example, welded). Tab(s) 305 can be attached at ends 301 and/or 302 (for example, at an edge of end 301) or alternatively can be attached away from an end 301. For example, tab 305 can be attached (for example, permanently secured) to a portion of the headband 300 that is spaced away from the end 301 (see FIGS. 7A-7B).

The one or more indicators of headband 300 can have various shapes and/or designs. For example, each of the one or more indicators can comprise notches 304a, 304b, 314a, 314b positioned along one or more edges of the elastic segment 303. The indicators of headband 300 can comprise notches 304a, 304b having a half-circle shape (see FIG. 7A), and/or notches 314a, 314b having a rectangular or square shape (see FIG. 7B). Additionally or alternatively, the one or more indicators of headband 300 can comprise notches having a different shape (for example, triangle or v-shaped). Notches 304a, 304b, 314a, 314b can be positioned closer to the first end 301 than to the second end 302 (see FIGS. 7A-7B). As discussed more below, when the headband 300 is wrapped around a portion of a user's body (for example, a forehead of the user) the location of the second end 302 with reference to one or more of the notches 304a, 304b, 314a, 314b can inform a user or a caregiver how much to stretch the headband 300 prior to securing the headband 300 to the portion of the user's body in order to achieve a desired or optimal force and/or pressure.

With reference to FIGS. 7A-7B, headband 300 can include one, two, three, four, five, six, seven, or eight or more notches 304a, 304b, 314a, 314b. As another example, headband 300 can include one or more pairs of notches 304a and 304b or notches 314a and 314b positioned along edges (for example, opposing edges) of the elastic segment 303. For example, headband 300 can include one, two, three, four, five, six, seven or eight or more pairs of notches 304a and 304b or notches 314a and 314b positioned along opposing edges of elastic segment 303. As another example, headband 300 can include a first pair of notches including a notch 304a positioned along a first edge of headband 300 and a corresponding notch 304b positioned along a second edge (opposite the first edge) of headband. In some cases, such first and second notches 304a, 304b are aligned (for example, vertically with reference to the orientation shown in FIG. 7A). With reference to FIG. 7A and 7B, headband 300 can include one or more notches 304a, 314a positioned along a first edge of the elastic segment 303 and additionally or alternatively include one or more notches 304b, 314b positioned along a second edge of the elastic segment 303.

In addition or as an alternative to notches 304a, 304b, 314a, 314b, headband 300 can include indicators that are defined by hatched or shaded regions or lines on portions of the elastic segment 303. For example, with reference to FIG. 7C, headband 300 can include one or more hatched or shaded regions or lines 324 which extend from one edge of the elastic segment 303 to an opposite edge of the elastic segment 303. The hatched or shaded regions or lines 324 can comprise a rectangular shape, among other shapes (see FIG. 7C). As discussed more below, when the headband 300 is wrapped around a portion of a user's body (for example, a forehead of the user) the location of the second end 302 with reference to one or more of the hatched or shaded regions or lines 324 can inform a user or a caregiver how much to stretch the headband 300 prior to securing the headband 300 to the portion of the user's body in order to achieve a desired or optimal force and/or pressure.

With reference to FIG. 7A, the one or more notches 304a can be positioned along an edge of the elastic segment 303 and spaced from one another by various spacings. Further, the one or more notches 304b can be positioned along an opposite edge of the clastic segment 303 and spaced from one another by various spacings. With reference to FIG. 7B, the one or more notches 314a can be positioned along an edge of the elastic segment 303 and spaced from one another by various spacings. Further, the one or more notches 314b can be positioned along an opposite edge of the elastic segment 303 and spaced from one another by various spacings. For example, centers of the notches 304a, 304b, 314a, 314b can be spaced apart by a distance S2 (see FIGS. 7A-7B). Further, ends of the notches 304a, 304b, 314a, 314b can be spaced apart by a distance S1 (see FIGS. 7A-7B). With reference to FIG. 7C, centerlines of the hatched or shaded regions or lines 324 can be spaced apart by distance S2, and/or leading and trailing edges of the hatched or shaded regions or lines 324 can be spaced apart by distance S1. The spacings S1 and/or S2 between any two consecutive notches 304a, 314a, and/or notches 304b, 314b can be selected so that the headband 300 exerts a desired or optimal force and/or pressure when stretched an amount equal to such spacing S1 and/or S2 (for example, where the headband 300 is wrapped around a forehead and end 302 is stretched beyond and/or along end 301 so as to position end 302 within a second consecutive notch 304a). For example, when headband 300 is stretched an amount equal to such spacing S1 and/or S2, the headband 300 can apply a force of 0.060 lbf, 0.120 lbf, 0.180 lbf, 0.240 lbf, 0.300 lbf, 0.360 lbf, 0.420 lbf, or 0.480 lbf on a sensor 100 and/or a forehead of a user, or any value therebetween, or any range bounded by any combination of these values, although values outside these values or ranges can be used in some cases. As another example, when headband 300 is stretched an amount equal to such spacing S1 and/or S2, the headband 300 can apply a pressure of 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi) on a sensor 100 and/or a forehead of a user, or any value therebetween, or any range bounded by any combination of these values, although values outside these values or ranges can be used in some cases.

With reference to FIGS. 7A-7B, the depth of notches 304a, 304b, 314a, 314b can be limited to a certain ratio or percentage of the width W of the elastic segment 303 to reduce the level of non-uniformity in the force and/or pressure applied by the band 300 (for example, the inward force applied to a physiological sensor 100 on a user's forehead when the band 300 is secured thereto) and/or to minimize loss of strength of the band 300. For example, the depth of notches 304a, 304b, 314a, 314b can be less than or equal to 50%, less than or equal to 45%, less than or equal to 40%, less than or equal to 35%, less than or equal to 30%, less than or equal to 25%, less than or equal to 20%, less than or equal to 15%, less than or equal to 10%, less than or equal to 5%, less than or equal to 4%, less than or equal to 3%, or less than or equal to 2% of the width W of the elastic segment 303, or any value therebetween, or any range bounded by any combination of these values, although values outside these values or ranges can be used in some cases.

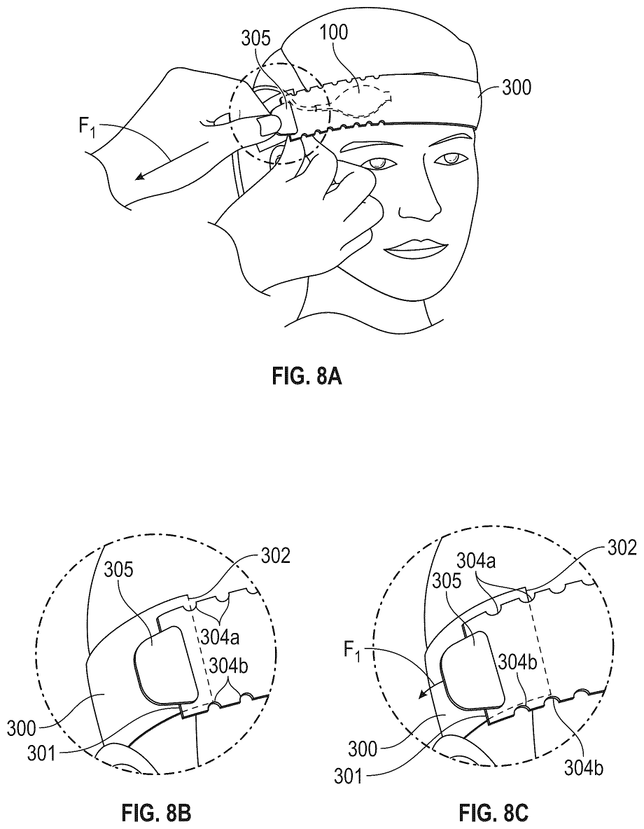

FIGS. 8A-8C illustrate an exemplary method of securing a headband 300 around a physiological sensor 100 and around a forehead of a user. While a forehead is illustrated in FIGS. 8A-8C, the method(s) of adjusting the length of the headband 300 to achieve a desired or optimal amount of force and/or pressure on the physiological sensor 100 and/or a portion of the user's body proximate the sensor 100 are applicable to other locations of a user's body. For example, the method(s) discussed below are equally applicable where the headband 300 is attached around a physiological sensor 100 on a user's foot, arm, stomach, chest, thigh, ankle, wrist, or any other location on a user's body.

FIG. 8A illustrates headband 300 having notches 304a, 304b located along opposing edges of the clastic segment 303. As discussed previously, headband 300 can include a plurality of notches 304a, 304b which can be spaced apart by a given spacing (S1 and/or S1). Further, the plurality of notches 304a, 304b can be positioned closer to one end of headband 300 (and/or elastic segment 303) than to another (for example, closer to end 301 than to end 302). As also discussed, the notches 304a, 304b can assist a wearer and/or caregiver in securing the headband 300 around physiological sensor 100 and a portion of the wearer's body (for example, forehead) such that an optimal force and/or pressure is applied and more accurate physiological measurements can be obtained.

As shown in FIG. 8A, a wearer and/or caregiver can begin by wrapping the headband 300 around a portion of the wearer's body (for example, forehead). The wearer and/or caregiver can hold an end 302 of the headband 300 (or elastic segment 303) and an opposite end 301 of the headband 300 and/or tab 305 (where headband 300 includes such tab 305). Prior to stretching the headband 300, the wearer and/or caregiver can align a first notch 304a and/or 304b with end 302 of the headband 300 such that the edge of end 302 of the headband 300 is positioned within a space defined by a first notch 304a and/or 304b. Use of the term “first” notch is intended to mean the notch 304a, 304b that are closest to the edge of end 302 after the headband 300 is wrapped around the portion of the wearer's body but not yet stretched. For example, when headband 300 is wrapped around a wearer's forehead, the circumference of the wearer's forehead can dictate which one of the plurality of indicators 304a and/or 304b the edge of end 302 is closest to prior to stretching of headband 300. The “first” notch 304a and/or 304b can be the one of the plurality of notches 304a and/or 304b that the edge of end 302 is closest to prior to stretching of the headband 300. For example, where notches 304a, 304b are positioned along edges of elastic segment 303, the “first” notch 304a and/or 304b can be the one that edge of end 302 falls within prior to stretching of the headband 300. In some cases, aligning both of the “first” notches 304a, 304b with the edge of end 302 can ensure that ends 301, 302 are properly aligned such that headband 300, when subsequently stretched, can apply a uniform force and/or pressure to the portion of the wearer's body that headband 300 is wrapped around.

After the alignment of the first notch 304a and/or 304b with the edge of end 302 takes place, the wearer and/or caregiver can stretch the headband 300 by holding end 302 of the headband 300 and pulling (see “F₁” in FIGS. 8A and 8C) the opposite end 301 and/or the tab 305 until the edge of end 302 is positioned within the space defined by a second notch 304a and/or 304b. Use of the term “second” notch 304a, 304b is meant to convey the “next” and/or “consecutive” notch 304a, 304b that is positioned farther from end 301 and/or tab 305 than the “first” notch 304a, 304b discussed above (for example, spaced apart from the first notch 304a, 304b by spacing S1 and/or S2). Similar to that discussed with reference to the first notch 304a and/or 304b, aligning both of the second notches 304a, 304b with the edge of end 302 can ensure that ends 301, 302 are properly aligned such that headband 300 can apply a uniform (or substantially uniform) force and/or pressure to the portion of the wearer's body that headband 300 is wrapped around.

After or during the alignment of the edge of end 302 with one or both of the second notches 304a, 304b, the headband 300 can be secured in place, for example, by attaching a portion of end 301 to end 302 and/or by attaching a portion of tab 305 to end 302. Such attachment can be by an adjustable closure mechanism, such as a hook and loop closure mechanism or an adhesive securement mechanism, as discussed elsewhere herein.

As discussed previously, the spacing S1 and/or S2 between notches 304a along an edge of the elastic segment 303 can be selected so that a predetermined incremental force is applied on the physiological sensor 100 when the headband 300 is stretched (and later secured in place) a distance or amount equal to such spacing S1 and/or S2. More specifically, spacing's S1 and/or S2 can be selected so that the extension (for example, via stretching) of end 301 beyond end 302 and alignment of the edge of end 302 with the “second” (for example, consecutive) notch(es) 304a, 304b can apply a predetermined incremental force and/or pressure on physiological sensor 100 and/or the portion of the user's body proximate the sensor 100. Thus, the stretching of headband 300 a distance equal to the spacing S2 between centers of first and second indicators 304a (and subsequent securement of the headband 300 in place) can apply a predetermined, predictable force on physiological sensor 100 and/or a predetermined, predictable pressure on the portion of the wearer's body proximate the sensor 100. Such predetermined force and/or pressure can be achieved by selecting an appropriate ratio or percentage of the spacings S2, S2 with respect to the length of the headband 300 (or clastic segment 303). In some cases, the spacing S1 and/or S2 between notches 304a, 304b is equal to a certain percentage of the length of elastic segment 303. For example, spacing S1, S2 can be 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, or 10% of a length of the clastic segment 303, or any value therebetween, or any range bounded by any combination of these values, although values outside these values or ranges can be used in some cases. For example, where the elastic segment 303 has a length of 25 inches and the spacing S1, S2 is equal to 0.7 inch, the spacing S1, S2 is 2.8% of the length of the elastic segment 303. In some cases, a ratio of the length of the elastic segment 303 to the spacing S2 and/or S2 to ensure that a desired incremental force and/or pressure results from stretching the elastic segment 303 by a value equal to spacing S1 and/or S2. For example, the ratio of the length of the elastic segment 303 to the spacing S1 and/or S2 can be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or any value therebetween, or any range bounded by any combination of these values, although values outside these values or ranges can be used in some cases. As another example, the ratio of the length of the elastic segment 303 to the spacing S1 and/or S2 can be between 20 and 30, between 20 and 40, between 20 and 50, between 30 and 40, between 30 and 50, or any value therebetween, or any range bounded by any combination of these values, although values outside these values or ranges can be used in some cases. As another example, the ratio of the length of the elastic segment 303 to the spacing S1 and/or S2 can be at least 20, at least 25, at least 30, at least 35, at least 40, at least 45, or at least 50, or any value therebetween, or any range bounded by any combination of these values, although values outside these values or ranges can be used in some cases.

As discussed below, the headband 300 (or the clastic segment 303 of headband 300) can have a variety of lengths. In some cases, where the elastic segment 303 is 25 inches, the spacing S1 and/or S2 can be 0.1 inch, 0.2 inch, 0.3 inch, 0.4 inch, 0.5 inch, 0.6 inch, 0.7 inch, 0.8 inch, 0.9 inch, 1 inch, 1.1 inch, 1.2 inch, 1.3 inch, 1.4 inch, 1.5 inch, 1.6 inch, 1.7 inch, 1.8 inch, 1.9 inch, or 2 inch, or any value therebetween, or any range bounded by any combination of these values, although values outside these values or ranges can be used in some cases. Keeping the spacing S1 and/or S2 within a certain percentage of the length of the headband 300 (and/or clastic segment 303) and/or keeping the ratio of the length of the elastic segment 303 to the spacing S1 and/or S2 can ensure that the stretching of headband 300 by an amount equal to the spacing S1 and/or S2 and subsequent securement of the headband 300 in place applies an incremental force that is within a desired or optimal comfort range. For example, keeping the spacing S1 and/or S2 within a certain percentage of the length of the headband 300 (and/or elastic segment 303) and/or keeping the ratio of the length of the elastic segment 303 to the spacing S1 and/or S2 can ensure that the stretching of end 301 beyond end 302 (when headband 300 is wrapped around a portion of the wearer's body) and subsequent securement of the headband 300 in place applies an incremental force that is within a desired or optimal comfort range. For example, stretching of the headband 300 by one additional/incremental notch 304a, 304b can result in the application of a force on the physiological sensor 100 that is between 0.120 lbf and 0.240 lbf. In some cases, stretching of the headband 300 by one additional/incremental indicator 304a, 304b can result in the application of a pressure on the region of the wearer's body proximate to the physiological sensor 100 and/or the sensor 100 that is between 10 mmHg (0.193 psi) and 20 mmHg (0.386 psi), which can reduce patient discomfort.

While FIGS. 8A-8C and the methods of securing/stretching headband 300 around a forehead has been shown and described above with reference to notches 304a, 304b, such figures and description is equally applicable to indicators 314a, 314b and/or 324. Additionally, while FIGS. 8A-8C illustrate alignment and/or positioning of the edge of end 302 within the space defined by indicators 304a, 304b (for example, the space defined by the half-circle notch), such alignment and/or positioning of the edge of end 302 could alternatively be within the portions of the edge of the elastic segment 303 between the half-circle notch indicators 304a, 304b. Further, the alignment and/or positioning of the edge of end 302 could alternatively be within the hatched and/or shaded regions or lines of indicators 324 and/or between these indicators 324.

With reference to FIGS. 7A-7C, the spacing S2 between centers of indicators 304a, 304b can be equal to, less than, or greater than the spacing S1 between ends of indicators 304a, 304b.

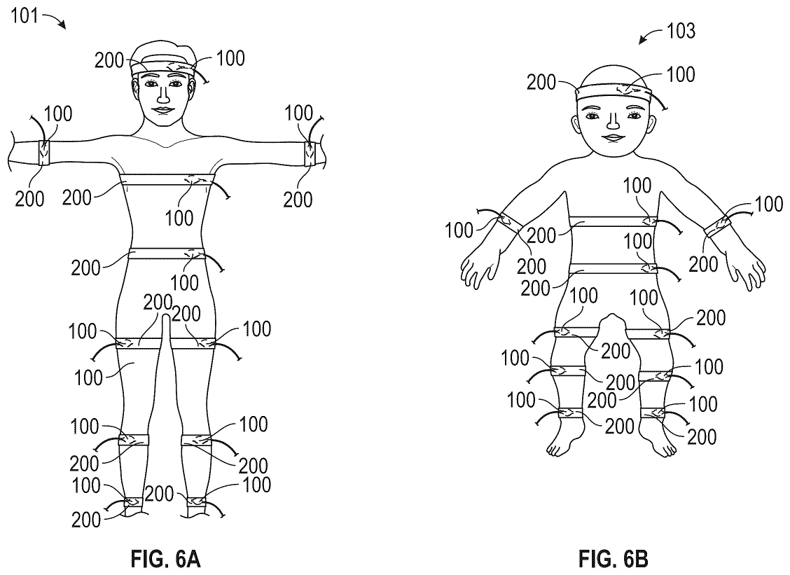

While the headbands discussed herein (for example, headbands 200, 300) have been at least partially discussed with reference to application to a wearer's head and/or forehead, the headbands, indicators, and/or limiters discussed herein can be useful in areas of a user's body other than the forehead. For example, as shown in FIGS. 6A-6B, the headband 200 including the indicators 202, 204, 220, and/or limiters 206 can surround, be placed over, and/or at least partially secure physiological sensors 100 to forearm regions, chest regions, abdominal regions, thigh regions, calf regions, foot regions, ankle regions, and/or renal regions, among others on an adult patient 101 and/or a neonatal patient 103. Similarly, headband 300 and/or sensor 100 can be placed and/or secure to and/or around non-forehead regions of a user, such as forearm regions, chest regions, abdominal regions, thigh regions, calf regions, foot regions, ankle regions, and/or renal regions, among others on an adult patient 101 and/or a neonatal patient 103. Thus, the headbands 200, 300 discussed herein can be used to apply pressure to sensors 100 and to underlying, adjacent, proximate skin of a wearer 101, 103 to provide for more accurate measurements as discussed previously.

The headbands 200, 300 (or portions thereof, such as the elastic segment 203, 303) discussed herein can be made of any suitable fabric. The elastic segment 203, 303 can be a braided clastic, knitted elastic, and/or woven elastic, for example. As another example, the clastic segment 203, 303 of the headband 200, 300 can comprise nylon and/or spandex. As another example, the clastic segment 203, 303 of the headband 200, 300 can comprise warp knit tricot nylon and/or spandex. As another example, the elastic segment 203, 303 can comprise a certain percentage of spandex and/or nylon. For example, the elastic segment 203, 303 can comprise between 1% and 20% spandex and between 80% and 99% nylon. For example, the clastic segment 203, 303 can comprise 1%, 2%, 3%, 4%, 5%, 6%, 7%, 8%, 9%, 10%, 11%, 12%, 13%, 14%, 15%, 16%, 17%, 18%, 19%, or 20% spandex, or any value therebetween, or any range bounded by any combination of these values, although values outside these values or ranges can be used in some cases. In some cases, the elastic segment 203, 303 can comprise a certain percentage of nylon having a certain thickness and/or linear density. For example, the elastic segment 203, 303 can comprise 5%, 6%, 7%, 8%, 9%, 10%, 11%, 12%, 13%, 14%, 15%, 16%, 17%, 18%, 19%, or 20% of nylon having 15 Denier, or any value therebetween, or any range bounded by any combination of these values, although values outside these values or ranges can be used in some cases. For example, the elastic segment 203 can comprise 75%, 76%, 77%, 78%, 79%, 80%, 81%, 82%, 83%, 84%, 85%, 86%, 87%, 88%, 89%, 90%, 91%, 92%, 93%, 94%, or 95% of nylon having 70 Denier, or any value therebetween, or any range bounded by any combination of these values, although values outside these values or ranges can be used in some cases. As an example, the elastic segment 203 can comprise between 5-10% spandex, between 5-15% nylon having 15 Denier, and between 75-90% nylon having 70 Denier. As another example, the elastic segment 203 can comprise 7-10% spandex, between 9-12% nylon having 15 Denier, and between 80-82% nylon having 70 Denier. As another example, the clastic segment can comprise 8.3% spandex, 10.5% nylon having 15 Denier, and 81.2% nylon having 70 Denier.

In some cases, the elastic segment 203, 303 can have a thickness of between 0.020 and 0.100 inches. As another example, the elastic segment 203, 303 can have a thickness of between 0.070 and 0.090 inches. As another example, the elastic segment 203, 303 can have a thickness of 0.080 inch. In some cases, the elastic segment 203, 303 can have a length of 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, 20 inches, 21 inches, 22 inches, 23 inches, 24 inches, 25 inches, 26 inches, 27 inches, 28 inches, 29 inches, 30 inches, 31 inches, 32 inches, 33 inches, 34 inches, or 35 inches, or any values therebetween, or any range bounded by any combination of these values, although values outside these values or ranges can be used in some cases.

In some cases, the elastic segment 203, 303 can have a width that is 0.25 inch, 0.5 inch, 0.75 inches, 1 inch, 1.25 inches, 1.5 inches, 1.75 inches, 2 inches, 2.25 inches, 2.5 inches, 2.75 inches, 3 inches, 3.25 inches, 3.5 inches, 3.75 inches, 4 inches, 4.25 inches, 4.5 inches, 4.75 inches, or 5 inches, or any values therebetween, or any range bounded by any combination of these values, although values outside these values or ranges can be used in some cases. For example, the elastic segment 203, 303 can have a width of 2 inches. In some cases, the width of the elastic segment 203, 303 can be a certain percentage of the length of the elastic segment in order to lower a distance of stretch per length required for a given force applied to the headband 200, 300. For example, the width of the elastic segment 203 can be 1%, 2%, 3%, 4%, 5%, 6%, 7%, 8%, 9%, 10%, 11%, 12%, 13%, 14%, 15%, 16%, 17%, 18%, 19%, or 20% of the length of the elastic segment 203, 303, or any values therebetween, or any range bounded by any combination of these values, although values outside these values or ranges can be used in some cases. In some cases, the weight of the elastic segment 203, 303 can be 1, 2, 3, 4, 5, 6, 7, 8, 9, 10, 11, 12, 13, 14, 15, 16, 17, 18, 19, or 20 oz/sq. yd., or any values therebetween, or any range bounded by any combination of these values, although values outside these values or ranges can be used in some cases. For example, the elastic segment 203, 303 can have a weight of 7.25 oz/sq. yd.

The elastic segment 203, 303 of the headband 200, 300 can be of one homogenous material composition and/or can be uniform in cross-section. Advantageously, having the clastic segment 203, 303 be one homogenous material composition can allow the headband 200, 300 to stretch in the same manner throughout the entirety of the headband 200, 300. For example, where the elastic segment 203, 303 extends from one end of the headband 200, 300 to an opposite end of the headband 200, 300 and is made of one homogenous material composition, the headband 200, 300 can stretch uniformly through the entirety of its length. Such uniformity in stretching along the headbands 200, 300 length can allow one or more indicators and/or limiters discussed herein to be positioned or placed along the entire length of the headband 200, 300 in order to provide visual indication of the headband's 200, 300 stretch from different viewpoints. For example, the indicators and/or limiters discussed herein can be positioned in a middle portion of the headband 200, 300 and/or locations between the middle portion and ends of the headband 200, 300. When the headband 200, 300 is secured to a wearer's forehead, for example, the indicators and/or limiters can appear at or proximate to side regions along the wearer's forehead in addition or alternative to front or back portions of the wearer's forehead. Where the elastic segment 203, 303 of the headband 200, 300 extends along the entire length of the headband 200, 300 and is of one homogenous material composition, the indicators can be placed along the entire length of the headband 200, 300 and can stretch in unison or uniformly with one another, for example.

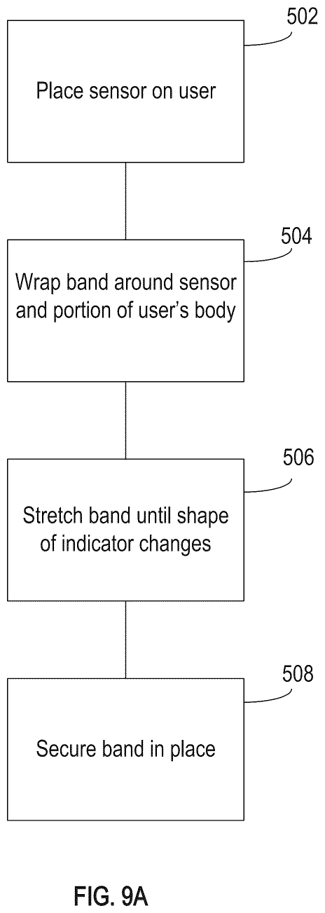

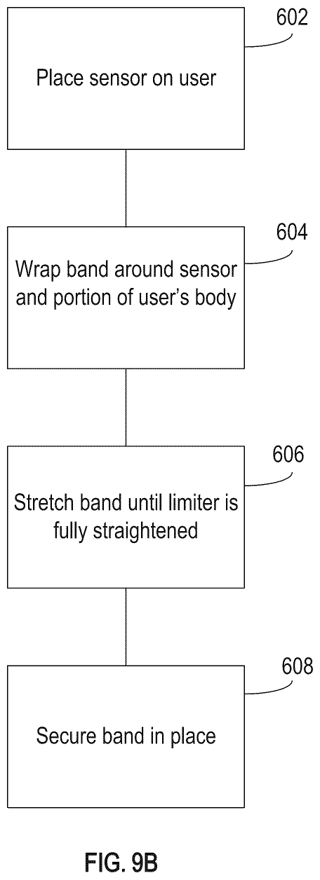

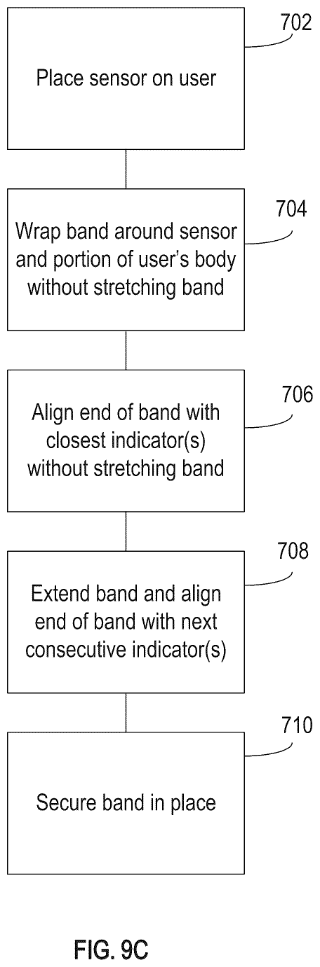

FIGS. 9A-9C illustrate exemplary methods of securing a band around a sensor and a portion of a user's body and extending (for example, stretching) the band so as to exert a desired or optimal force and/or pressure. FIG. 9A illustrates an exemplary method for securing headband 200 having indicators 202, 204, 220 around a sensor and portion of a user's body. As discussed above, headband 200 can have indicators 202, 204 that comprise a first shape when the headband 200 is in an un-stretched position and a second (for example, different) shape when the headband 200 is in an optimally-stretched position which results in the headband 200 applying a predetermined force and/or pressure. As also discussed above, headband 200 can alternatively or additionally have indicators 220 which comprise a pattern and/or group of shapes that changes in orientation, size, and/or shape when the headband 200 is extended from a first, un-stretched position to a second, optimally-stretched position which results in the headband 200 applying a predetermined force and/or pressure. The dimensions, orientation, layout, and/or shape of the shapes and/or pattern or group of shapes which define the indicators 202, 204, 220 can be configured such that, the “optimally-stretched” position (for example, the position which produces the second shape) can result in the application of a force and/or pressure on the sensor and/or portion of the user's body proximate, adjacent, and/or underneath the sensor that increases the accuracy of physiological measurements and remains within a comfortable range, as discussed above.

Turning to FIG. 9A, a user or caregiver can begin at step 502 by placing a sensor 100 on a portion of a user's body (for example, a forehead of user). The user or caregiver can, at step 504, thereafter wrap band 200 around the sensor 100 and the portion of the user's body so as to at least partially cover the sensor 100. Alternatively, the sensor 100 can be placed on the portion of the user's body simultaneously with the wrapping of the band 200 around sensor 100 and the portion of the user's body. At step 506, the user or caregiver can stretch band 200 until indicator(s) 202, 204, 220 change from the first shape/design to the second shape/design. For example, the user or caregiver can stretch band 200 until indicator(s) 202 change from the rectangular shape to the square shape (see FIGS. 2A-2B). As another example, the user or caregiver can stretch band 200 until indicator(s) 204 change from the ellipse to the circle (see FIGS. 3A-3B). As another example, the user or caregiver can stretch band 200 until the indicator(s) 220 changes from the first, rectangular pattern(s) to the second, square pattern(s). At step 508, when such transformation occurs, the user or caregiver can secure the band 200 in place around the sensor 100 and portion of the user's body. Such securement can occur by attaching a portion of the band 200 to itself, for example, via a hook and loop fastener mechanism and/or an adhesive (for example, a removable adhesive) mechanism, among other types of securement.

FIG. 9B illustrates an exemplary method for securing headband 200 having limiter 206 around a sensor and portion of a user's body. As discussed above, limiter 206 can include a loose segment 210 which connects to portions of the headband 200. Turning to step 602, a user or caregiver can begin by placing a sensor 100 on a portion of a user's body (for example, a forehead of user). The user or caregiver can, at step 604, thereafter wrap band 200 around the sensor 100 and the portion of the user's body so as to at least partially cover the sensor 100. Alternatively, the sensor 100 can be placed on the portion of the user's body simultaneously with the wrapping of the band around sensor 100 and the portion of the user's body. At step 606, the user or caregiver can stretch band 200 until limiter 206 (and/or the loose segment 210) is fully straightened or “taut”. As discussed above, the limiter 206 can comprise a different material than the headband 200 and can be substantially non-elastic such that is does not stretch along with the headband 200 when the headband 200 is stretched. Once the limiter 206 is fully straightened, at step 608, the user or caregiver can secure the band 200 in place around the sensor 100 and portion of the user's body. Such securement can occur by attaching a portion of the band 200 to itself, for example, via a hook and loop fastener mechanism and/or an adhesive (for example, a removable adhesive) mechanism, among other types of securement.

FIG. 9C illustrates an exemplary method for securing band 300 having one or more of indicator(s) 304a, 304b, 314a, 314b, 324 around a sensor and portion of a user's body. Turning to step 702, a user or caregiver can begin by placing a sensor 100 on a portion of a user's body (for example, a forehead of user). The user or caregiver can, at step 704, thereafter wrap band 300, without stretching the band 300, around the sensor 100 and the portion of the user's body to at least partially cover the sensor 100. Alternatively, the sensor 100 can be placed on the portion of the user's body simultaneously with the wrapping of the band 300 around sensor 100 and the portion of the user's body. With reference to FIG. 9C and 8B, at step 706 a user or caregiver can then align an end of band 300 (such as end 302) with the closest indicator(s) (such as notches 304a, 304b) without stretching band 300. For example, end 302 can be at least partially positioned within a space defined by a notch 304a or 314a along a first edge and/or a notch 304b or 314b along a second edge of headband 300. Thereafter, at step 708, the user or caregiver can stretch band 300 (for example, by exerting force F₁ on tab 305 and/or end 301) until end 302 is aligned with the next, consecutive notch(s) 304a, 304b (see FIG. 8C). For example, the user or caregiver can stretch band 300 until end 302 is at least partially positioned within a space defined by one or more of the next, consecutive notches 304a, 304b. After step 708, the user or caregiver can, at step 710, secure the band 300 in place around the sensor 100 and portion of the user's body. Such securement can occur by attaching a portion of the band 300 to itself, for example, via a hook and loop fastener mechanism and/or an adhesive (for example, a removable adhesive) mechanism, among other types of securement. As discussed elsewhere herein, the spacing of the notches 304a or notches 314a along a first edge of headband 300 and/or notches 304b or 314b along a second, opposite edge of headband 300, can be selected so that the stretching and alignment of the next, consecutive notch causes the headband 300 to exert an incremental desired or optimal force and/or pressure on the sensor 100 and/or region of the user's body proximate the sensor 100. For example, the spacing S1 and/or S2 of the notches 304a from one another along an edge of headband 300 can be such that the stretching of end 302 a distance equal to such spacing results in an incremental force of 0.060 lbf, 0.120 lbf, 0.180 lbf, 0.240 lbf, 0.300 lbf, 0.360 lbf, 0.420 lbf, or 0.480 lbf, or any value therebetween, or any range bounded by any combination of these values, although values outside these values or ranges can be used in some cases. As another example, the spacing S1 and/or S2 of the notches 304a from one another along an edge of headband 300 can be such that the stretching of end 302 a distance equal to such spacing results in an incremental pressure of 5 mmHg (0.0967 psi), 10 mmHg (0.193 psi), 15 mmHg (0.290 psi), 20 mmHg (0.386 psi), 25 mmHg (0.483 psi), 30 mmHg (0.580 psi), 35 mmHg (0.677 psi), or 40 mmHg (0.773 psi), or any value therebetween, or any range bounded by any combination of these values, although values outside these values or ranges can be used in some cases.

### Additional Considerations

Although this disclosure has been disclosed in the context of certain examples, it will be understood by those skilled in the art that the present disclosure extends beyond the specifically disclosed examples to other alternative examples and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the examples may be made and still fall within the scope of the disclosure. Accordingly, it should be understood that various features and aspects of the disclosure can be combined with or substituted for one another in order to form varying modes of the disclosed.

Features, materials, characteristics, or groups described in conjunction with a particular aspect, or example are to be understood to be applicable to any other aspect, or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples of systems. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that the actual steps taken in the processes illustrated and/or disclosed may differ from those shown in the figures. Depending on the system, certain of the steps described above may be removed, others may be added. Furthermore, the features and attributes of the specific examples disclosed above may be combined in different ways to form additional examples of systems, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain systems include, while other systems do not include, certain features, elements, and/or steps. Thus, such conditional language is not generally intended to imply that features, elements, and/or steps are in any way required for one or more systems or that one or more systems necessarily include logic for deciding, with or without user input or prompting, whether these features, elements, and/or steps are included or are to be performed in any particular system.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. Additionally, as used herein, “gradually” has its ordinary meaning (e.g., differs from a non-continuous, such as a step-like, change).

The scope of the present disclosure is not intended to be limited by the specific disclosures of the systems in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. (canceled)

2. A method of securing a band around a physiological sensor on a portion of a user's body, the method comprising:
wrapping the band around the portion of the user's body to at least partially cover the physiological sensor, wherein the band comprises:
an elastic segment having a first end and a second end opposite the first end, the elastic segment having a first length when in an unstretched state; and
a first indicator and a second indicator, the first indicator and second indicator each comprising a pair of notches, the first indicator and second indicator spaced apart from one another and positioned along the elastic segment closer to the first end than to the second end, wherein the first indicator is positioned closer to the first end than the second indicator;
at least partially aligning the first indicator with the second end of the elastic segment while the band is in an unstretched state;
stretching the band to a second length, the second length being greater than the first length;
at least partially aligning the second indicator with the second end of the elastic segment, alignment of the second indicator with the second end indicative of a first pressure exerted on the portion of the user's body; and
securing the band to the portion of the user's body.

3. The method of claim 2, wherein the band further comprises a tab connected to the elastic segment proximate the first end, and wherein the step of securing the band to the portion of the user's body comprises securing the tab to a portion of the elastic segment.

4. The method of claim 2, wherein the elastic segment comprises a first edge and a second edge opposite the first edge, each of the first and second edges extending between the first and second ends of the elastic segment, and wherein each notch of the first and second indicators are positioned along the first edge or the second edge.

2. The method of claim 2, wherein the step of at least partially aligning the first indicator with the second end while the band is in an unstretched state comprises positioning the portion of the second end within a space defined by the pair of notches of the first indicator.

5. The method of claim 5, wherein the band further comprises a third indicator, the third indicator comprising a pair of notches, the third indicator spaced apart from the first indicator and second indicator and positioned along the elastic segment closer to the first end than to the second end, wherein a distance between the first indicator and the third indicator along a length of the band is larger than a distance between the second indicator and the third indicator along a length of the band, the method further comprising at least partially aligning the third indicator with the second end, wherein alignment of the second end with the third indicator is indicative of a second pressure exerted on the portion of the user's body, wherein the second pressure is greater than the first pressure.

2. The method of claim 2, wherein the first and second indicators are spaced apart from one another by a distance, and wherein the distance is between 1% and 5% of a length of the elastic segment.

8. The method of claim 2, wherein the first pressure is between about 5 mmHg (0.0967 psi) and 15 mmHg (0.290 psi).

9. The method of claim 2, wherein the step of securing comprises engaging a hook and loop mechanism between an outer surface of the band an inner surface of the second end.
